(19) United States
(12) Patent Application Publication
(10) Pub. No.: US 2023/0117738 A1
(43) Pub. Date: Apr. 20, 2023

(54) GENERATION OF AUTOIMMUNE INHIBITORY T CELLS BY FIBROBLAST MEDIATED EDUCATION

(71) Applicant: FIGENE, LLC, Houston, TX (US)

(72) Inventor: Thomas ICHIM, San Diego, CA (US)

(21) Appl. No.: 17/905,197

(22) PCT Filed: Mar. 12, 2021
(86) PCT No.: PCT/US2021/022145

§ 371 Date: Aug. 29, 2022

Related U.S. Application Data
(60) Provisional application No. 62/989,118, filed on Mar. 13, 2020.

(51) Int. Cl. A61K 35/17; A61K 35/15; C12N 5/0783

(52) U.S. Cl. CPC A61K 35/17; A61K 35/15; C12N 5/0637; C12N 2501/2302; C12N 2501/2304; C12N 2501/2307; C12N 2502/1114; C12N 2502/1323

(57) ABSTRACT

Disclosed are means, methods, and compositions of matter useful for treatment of autoimmunity comprising exposing patient T cells fibroblasts possessing tolerogenic properties. In one embodiment peripheral blood mononuclear cells are cultured in the presence of CD73 selected fibroblasts alone or in the presence of interleukin-2, and/or IL-7, and/or TGF-beta, and/or PGE-2 for a period of time sufficient to endow tolerogenic properties. Said tolerogenic properties include ability to suppress adaptive or innate immune responses. In another embodiment the disclosure provides methods of generating antigen specific immune regulatory cells by culture of T cells together with fibroblasts in the presence of antigen to which specific immune regulation is desired.

This application claims priority to U.S. Provisional Patent Application Ser. No. 62/989,118, filed Mar. 13, 2020, which is incorporated by reference herein in its entirety.

### TECHNICAL FIELD

Embodiments of the disclosure encompass at least the fields of cell biology, molecular biology, and medicine.

### BACKGROUND

The immune response is known to possess incredible efficacy at neutralizing microscopic pathogens, whether bacteria, parasitic or viral, and to some extent cancer cells. In general, the complicated mechanisms for self-recognition are efficient and allow a strong response to be directed exclusively at eliminating foreign antigens. The regulation of self/non-self-discrimination, which is a critical function of the immune system, involves multiple mechanisms during the development and lifespan of adaptive immune cells such as T and B lymphocytes, as well as “semi-adaptive” immune cells such as NKT cells, gamma delta T cells, and NK cells [1].

An unfortunate medical reality is that in some situations, the immune system occasionally malfunctions and turns against the cells of the host, thereby provoking an autoimmune response. Autoimmunity typically occurs when antigen receptors on immune cells recognize specific self-antigens on host cells and initiate reactions that result in the destruction of the host cells. In some instances, the autoreactive lymphocytes survive longer and continue to induce apoptosis or otherwise eliminate host cells causing autoimmune diseases. Different mechanisms [2], have been described that prevent T lymphocytes from attacking self. These tolerance mechanisms act both on developing and mature T cells. For example, thymic positive selection skews the T cell repertoire to recognize self-MHC molecules and thus also enriches for auto-reactive T cells [3, 4]. On the other hand, thymic negative selection, which follows positive selection, eliminates auto-reactive T cells either by clonal deletion (death) or inactivation, (anergy). Whereas clonal deletion deals with high affinity T cells at the CD4+CD8+TCR^{high} maturation stage, clonal inactivation seems to work at lower affinity interaction possibly by the down-regulation of the TCR and the CD8 α-chain. However, these “anergic” T lymphocytes still have the ability to specifically respond to their antigen. It has been suggested that they actually might represent a population of regulatory T cells since they release IL-10 and TGF-beta upon stimulation. Thus, it is well-established that thymic (central) tolerance mechanisms do not eliminate all autoreactive T cells. Indeed, T cells reactive with self-antigens, such as myelin basic protein (MBP), insulin and glutamic acid decarboxylase (GAD), can be readily found in the periphery.

There is a need to generate antigen-specific immune suppressor cells, such as for treatment of multiple sclerosis and other autoimmune conditions.

### BRIEF SUMMARY

Certain embodiments of the disclosure relate to the area of immune modulation. Some embodiments relate to the use of cell populations, including populations comprising fibroblast cells, for expansion of immune regulator cells. Some embodiments relate to the utilization of allogeneic fibroblast cells for stimulation of autologous immune regulatory cells. Certain embodiments relate to the use of tolerogenic fibroblast cells for expansion of autologous immune regulatory cells.

Certain embodiments concern methods for generating an immune modulatory cell population. Some embodiments concern methods comprising contacting a first cell population with a second cell population. In some embodiments, the first or second cell population comprises a fibroblast population. In some embodiments, the first or second cell population comprises an immune cell population. The first cell population and/or the second cell population may be obtained from a subject. The subject may be in need of a therapy. In some embodiments, the contacting between the first cell population and the second cell population generates a third cell population. The third cell population may have immune modulatory properties.

Certain embodiments concern fibroblast cell populations. The fibroblasts may be isolated from any tissue, including bone marrow, adipose, placenta, tonsil, skin, hair follicle, omentum, peripheral blood, Wharton's jelly, or a combination thereof. The fibroblast cell population may be autologous, xenogeneic, or allogeneic with respect to a subject, including a subject in need of a therapy. The fibroblast population may express at least one of CD13, CD44, CD49b, CD73, CD90, CD105, or a combination thereof. In some embodiments, the fibroblast population lacks expression of at least one of CD14, CD34, CD45, HLA class II, SSEA-1, or a combination thereof. In some embodiments, the fibroblast population comprises cells capable of differentiating into bone, cartilage, and/or adipose tissue

In some embodiments, the fibroblast population is contacted with one or more compositions, which may be before the fibroblast population is contacted with a different cell population, such as a T cell population. The composition may be human chorionic gonadotropin (hCG). In some embodiments, the fibroblast population is contacted with hCG at a concentration of about 5 IU/mL, although other concentrations may be utilized, including 0.1-100 IU/mL and any range derivable therebetween.

In some embodiments, the fibroblast population produces FGF-1, FGF-2, TGF-beta, or a combination thereof. The fibroblast population may produce FGF-1 at a concentration of at least 2 ng/mL per million cells in the population. The fibroblast population may produce FGF-2 at a concentration of at least 1 ng/mL per million cells in the population. The fibroblast population may produce TGF-beta at a concentration of at least 10 ng/mL per million cells in the population. The fibroblast population may produce exosomes at a concentration of at least 10 ng/mL per million cells in the population. The exosomes may comprise the marker CD81 and/or membrane bound TGF-beta.

In some embodiments, the fibroblast population comprises a cell population derived from Wharton's Jelly that has been enzymatically digested, and the resulting cells have been centrifuged, plated onto fibronectin-coated plates in medium with 2% serum, wherein the fibroblast population was selected from cell colonies that adhere to the plates, and optionally wherein the fibroblast population has mortal, epithelioid morphology.

In some embodiments, the fibroblast population possesses an ability to inhibit secretion of at least one inflammatory cytokine. The cytokines may be any cytokine, including IFN-gamma, TNF-alpha, IL-2, IL-7, IL-12, IL-15, IL-17, IL-18, IL-21, IL-23, IL-27, IL-33, HMGB-1, TRAIL, or a combination thereof.

In some embodiments, the fibroblast population is cultured. The fibroblast population may be cultured for at least 1, 2, 3, 4, 5, 6, 7, 8, 9, 10, 11, 12, 13, 14, or more days. The fibroblast population may be maintained in an undifferentiated state, including when contacted with a second cell population.

Certain embodiments concern cell populations that comprise immune cells selected from the group consisting of B cells, T cells, innate lymphoid cells, natural killer cells, natural killer T cells, gamma delta T cells, macrophages, monocytes, dendritic cells, neutrophils, myeloid derived suppressor cells, or a combination thereof. The B cells may comprise CD5+ and/or CD5− B cells. The B cells may comprise naïve B cells and/or memory B cells. The T cells may comprise CD4+ and/or CD8+ T cells. The T cells may comprise CD28+ and/or CD28− T cells. The T cells may comprise memory T cells and/or naïve T cells. The T cells may express CD3. The T cells may comprise T regulatory cells, which may express CD25, CTLA-4, FoxP3, or a combination thereof.

The T cells may comprise Th1 cells. The Th1 cells may express CD4, CD94, CD119 (IFNγ R1), CD183 (CXCR3), CD186 (CXCR6), CD191 (CCR1), CD195 (CCR5), CD212 (IL-12Rβ1&2), CD254 (RANKL), CD278 (ICOS), IL-18R,MRP1, NOTCH3, TIM3, or a combination thereof. The T cells may comprise Th2 cells. The Th2 cells may be capable of secreting cytokines, such as IL-4, IL-5, IL-6, IL-9, IL-10, IL-13, or a combination thereof. The Th2 cells may express markers selected from the group consisting of CRTH2, CCR4, CCR3, and a combination thereof.

The innate lymphoid cells may be innate lymphoid cells 1, innate lymphoid cells 2, innate lymphoid cells 3, lymphoid tissue inducer cells, or a combination thereof. The innate lymphoid cell 1 may express T bet, respond to IL-12 by secretion of interferon gamma, and/or lack expression of perforin and CD56. The innate lymphoid cell 2 may produce IL-4 and/or IL-13. The innate lymphoid cell may produce IL-17a and/or IL-22. The lymphoid tissue inducer cells may comprise cells involved in the induction of memory T cells.

The macrophages may comprise M1 macrophages and/or M2 macrophages. The M1 macrophages may be capable of producing nitric oxide. The M2 macrophages may be capable of producing arginase.

The dendritic cells may comprise myeloid dendritic cells or lymphoid dendritic cells. The myeloid dendritic cells may be capable of stimulating Th1 immune responses in a mature state. The myeloid dendritic cells in said mature state may express substantially higher levels of CD80, CD86, CD40, or a combination thereof as compared to myeloid dendritic cells in an immature state. The lymphoid dendritic cells may be capable of producing interferon alpha.

Certain embodiments concern cell populations comprising peripheral blood mononuclear cells. Certain embodiments concern cell populations comprising buffy coat cells.

In certain embodiments, any cell population encompassed herein is cultured with amniotic fluid stem cells, including in a manner permitting cell to cell contact. The cell to cell contact is provided by culturing on tissue culture plates at a concentration sufficient to cover a surface of said tissue culture plate. In some embodiments, the ratio of cells in a cell population, including a population of peripheral blood mononuclear cells, to amniotic fluid stem cells is 1 to 1. In some embodiments, the ratio of cells in a cell population, including a population of T regulatory cells mononuclear cells, to amniotic fluid stem cells is 1 to 5. In certain embodiments, a cell population, which may comprise CD3 isolated T cells, is in a tissue culture at a 1:2 ratio with amniotic fluid stem cells in the presence of interleukin-2, VEGF, PGE-2, TGF-beta, at least one mTOR inhibitor, an anti-CD3 antibody, an anti-CD28 antibody, or a combination thereof for approximately 5-10 days, such as 5, 6, 7, 8, 9, or 10 days. The interleukin-2 may be added to the tissue culture at a concentration sufficient to induce expansion of T cells with immune modulatory activity. Such concentration may be 2 pg/mL to 100 ng/mL of tissue culture fluid. The mTOR inhibitor may comprise rapamycin, everolimus, or a combination thereof. The anti-CD3 antibody and/or the anti-CD28 antibody may be immobilized to a solid surface, such as a tissue culture plate and/or biocompatible bead.

In some embodiments, a first cell population (including any cell population herein) is contacted with a second cell population (including any cell population herein). The either or both cell populations may also be contacted with interleukin-2, including during the first cell population contacting the second cell population.

Certain embodiments concern subjects, including subjects in need of therapy. The subject may have, or be suspected of having, an autoimmune condition. The autoimmune condition may be, for example, multiple sclerosis.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIG. 1 shows fibroblasts enhancing generation of CD25 immune modulatory cells in the presence of IL-4 or IL-7. From left to right, the bars represent control, bone marrow-MSCs, adipose-MSCs, and fibroblasts.

FIG. 2 shows IL-2 enhances CD25 cell generation by fibroblasts. From left to right, the bars represent control, bone marrow-MSCs, adipose-MSCs, and fibroblasts.

FIG. 3 shows IL-2 and fibroblast enhancing generation of Tregs expressing FoxP3. From left to right, the bars represent control, bone marrow-MSCs, adipose-MSCs, and fibroblasts.

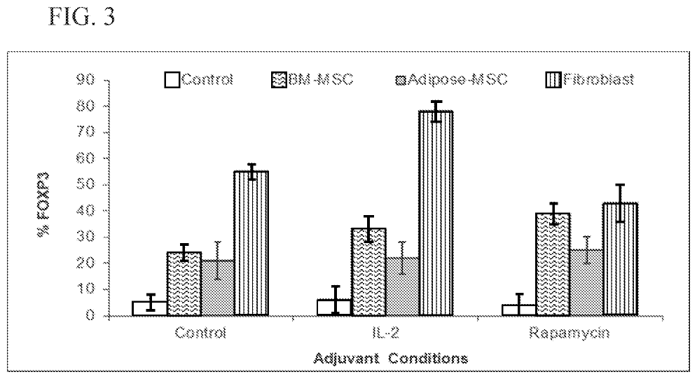

FIG. 4 shows IL-2 and fibroblast generated Tregs suppress T Cell proliferation.

FIG. 5 shows IL-2 and fibroblast Generated Tregs prevent experimental autoimmune encephalopathy (EAE).

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” or “approximately” is used according to its plain and ordinary meaning in the area of cell and molecular biology to indicate that a value includes the standard deviation of error for the device or method being employed to determine the value. The terms may refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, “allogeneic” refers to tissues or cells or other material from another body that in a natural setting are immunologically incompatible or capable of being immunologically incompatible, although from one or more individuals of the same species. Allogenic cells are cells of the same species that differ genetically from cells of a host.

“Autologous,” as used herein, refers to cells derived from the same subject and/or having the same genetic material.

As used herein, the terms “carrier” and “diluent” refer to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

As used herein, “cells”, which may be interchangeable with “cell population” or “population”, when in reference to a particular cell type, refers to at least one cell of the particular cell type. For example, fibroblast cells, which may be interchangeable with a fibroblast population or fibroblast cell population, refers to a plurality of cells that comprise, consist essentially of, or consist of fibroblasts.

As used herein, “cell line”, which may be interchangeable with “cell population”, refers to a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to seeding density, substrate, medium, growth conditions, and time between passaging.

As used herein, “conditioned medium” describes medium in which a specific cell or population of cells has been cultured for a period of time, and then removed, thus separating the medium from the cell or cells. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. In this example, the medium containing the cellular factors is conditioned medium.

As used herein, a “trophic factor” describes a substance that promotes and/or supports survival, growth, proliferation and/or maturation of a cell. Alternatively or in addition, a trophic factor stimulates increased activity of a cell.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Certain cell populations encompassed herein are “culture expanded populations”, meaning populations of cells whose numbers have been increased by cell division in vitro. This term may apply to stem cell populations and non-stem cell populations alike.

As used herein, “does not detectably express” or “lacks expression of” means that expression of a protein or gene or transcript cannot be detected by standard methods. In the case of cell surface markers, expression can be measured by, e.g., flow cytometry, using a cut-off values as obtained from negative controls (i.e., cells known to lack the antigen of interest) or by isotype controls (i.e., measuring nonspecific binding of the antibody to the cell). Thus, a cell that lacks expression of a marker appears similar to the negative control for that marker. For gene expression, a gene may lack expression if the presence of its mRNA cannot be visually detected on a standard agarose gel following standard PCR protocols. Conversely, in certain embodiments, a cell “expresses” the protein or gene or transcript if it can be detected by the same method.

“Fibroblasts” as used herein, intra alia, are cells of the disclosure from various tissues, selected for specific properties associated with regenerative activity. Tissues useful for the practice of the disclosure are generally tissues associated with regenerative activity. Said tissues include placenta, endometrial cells, Wharton's jelly, bone marrow, and adipose tissue. In certain embodiments, fibroblasts are cells selected for expression of the markers CD117, CD105, and comprising rhodamine 123 efflux activity, or a combination thereof. In some embodiments, fibroblasts are cells selected for expression of markers selected from the group consisting of Oct-4, CD-34, KLF-4, Nanog, Sox-2, Rex-1, GDF-3, Stella, and a combination thereof. Certain fibroblasts of embodiments herein possess enhanced expression of GDF-11. Selection of fibroblasts for expression of the markers may be performed by initial expression of proteins found on the membrane of the cells, which result in possessing other markers mentioned.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to bone. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 30, 35, 40, 45, 50, 55, 60, 65, 70, 75, 80, 85, 90, 95, or 100 or more.

The term “fibroblast-derived product” (also “fibroblast-associated product”), as used herein, refers to a molecular or cellular agent derived or obtained from one or more fibroblasts. In some cases, a fibroblast-derived product is a molecular agent. Examples of molecular fibroblast-derived products include conditioned media from fibroblast culture, microvesicles obtained from fibroblasts, exosomes obtained from fibroblasts, apoptotic bodies, apoptotic vesicles, nucleic acids (e.g., DNA, RNA, mRNA, miRNA, etc.) obtained from fibroblasts, proteins (e.g., growth factors, cytokines, etc.) obtained from fibroblasts, and lipids obtained from fibroblasts. In some cases, a fibroblast-derived product is a cellular agent. Examples of cellular fibroblast-derived products include cells (e.g., stem cells, hematopoietic cells, neural cells, etc.) produced by differentiation and/or de-differentiation of fibroblasts.

The term “passaging” refers to the process of transferring a portion of cells from one culture vessel into a new culture vessel.

### II. Methods and Compositions for Disease Treatment or Prevention of Autoimmune Diseases

Certain embodiments concern the enhancement or generation of immune cells by co-culture of a cell population with fibroblasts. In some embodiments, the cell population is obtained from a subject in need of therapy. The subject in need of therapy may be administered one or more of the cell populations generated in embodiments herein. In some embodiments, the co-culture generates immune modulatory cells, such as immune modulatory T cells. The cells may be used in treating and/or preventing an autoimmune disease, such as multiple sclerosis. The disclosure provides means of expanding immune modulatory cells using fibroblast cells. In certain embodiment of the disclosure, fibroblast cells are grown together with a second cell population, such as immune cells and/or peripheral blood mononuclear cells, in a culture containing a suitable ratio. The ratio may be, for example, approximately 3:1, 2:1, 1:1, 1:2, or 1:3 fibroblasts to cells of the second cell population. The co-culture may be performed for 1, 2, 3, 4, 5, 6, 7, 8, 9, 10, 11, 12, 13, 14, 15, 16, 17, 18, 19, 20, 21, or more days. The co-culture may be in the absence or presence of one or more cytokines. In some embodiments, the cytokine comprises IL-2 and is present in the culture at about 35 ng/mL. In some embodiments, the cytokine comprises IL-4 and/or IL-7. In some embodiments, after culture, nonadherent cells are extracted and administered to a subject suffering from an autoimmune and/or inflammatory condition, The condition may be a neuroinflammatory condition. The condition may be multiple sclerosis. In certain embodiments, fibroblasts are selected for enhanced immune modulating potential. In one specific embodiment, fibroblasts are chosen for expression of CD73. Selection of these fibroblasts may be performed in order to attain an increased tolerogenic activity. In some embodiments, fibroblasts are utilized to generate T regulatory cells.

Certain embodiments provide the use of fibroblasts to generate cells that are Tregs, or appear to function similar to Tregs. The fibroblasts, in an embodiment, are incubated with cells, including any immune cell encompassed herein, from the diseased patient, and used to enhance generation of Treg formation. The role of Treg cells is universally associated with tolerance in conditions of natural tolerance such as in pregnancy [5, 6], transplantation tolerance [7-11], and ocular tolerance [12-21]. The functional relevance of Treg cells to preservation and/or initiation of tolerance is observed in conditions where administration of Tregs prevents pathology, such as in spontaneous abortion [22]. In some embodiments of the disclosure, Treg are generated ex vivo and administered in vivo with supplemental IL-2 to enhance in vivo expansion of the ex vivo generated T reg. Concentrations for clinical uses of aldesleukin could be used from the literature as described for other indications including heart failure [23], Wiskott-Aldrich syndrome [24], Graft Versus Host Disease [25, 26], lupus [27], type 1 diabetes [28-30] and are incorporated by reference. In some embodiments of the disclosure, administration of low doses of IL-2 in the form of aldesleukin every day at concentrations of 0.3×10⁶ to 3.0×10⁶ IU IL-2 per square meter of body surface area for 8 weeks, or in other embodiments repetitive 5-day courses of 1.0×10⁶ to 3.0×10⁶ IU IL-2. Various types of IL-2 may be utilized in embodiments herein. Examples of IL-2 variants, recombinant IL-2, methods of IL-2 production, methods of IL-2 purification, methods of formulation, and the like are well known in the art and can be found, for example, at least in U.S. Pat. Nos. 4,530,787, 4,569,790, 4,572,798, 4,604,377, 4,748,234, 4,853,332, 4,959,314, 5,464,939, 5,229,109, 7,514,073, and 7,569,215, each of which is herein incorporated by reference in their entirety for all purposes. In some embodiments of the disclosure, administration of estrogen is performed in order to assist in the expansion of Treg cells. The utilization of estrogen to advance expansion of Treg cells has previously been demonstrated and is incorporated by reference. For example, it is known that CD4(+)CD25(+) regulatory T cells are crucial to the maintenance of tolerance in normal individuals, such as in augmenting FoxP3 expression in vitro and in vivo [31].

### III. Fibroblasts and Cultured Cells

Aspects of the present disclosure comprise cells useful in therapeutic methods and compositions. Cells disclosed herein include, for example, fibroblasts, stem cells (e.g., hematopoietic stem cells or mesenchymal stem cells), endothelial progenitor cells, immune cells (e.g. B cells, T cells, innate lymphoid cells, natural killer cells, natural killer T cells, gamma delta T cells, macrophages, monocytes, dendritic cells, neutrophils, or myeloid derived suppressor cells), and peripheral blood mononuclear cells. Cells of a given type (e.g., fibroblasts) may be used alone or in combination with cells of other types. For example, a cell population may be isolated and provided to a subject alone or in combination with one or more stem cells. In some embodiments, disclosed herein are cell populations capable of preventing or treating an autoimmune disease, such as multiple sclerosis. In some embodiments, fibroblasts of the present disclosure are adherent to plastic. In some embodiments, fibroblasts express CD73, CD90, and/or CD105. In some embodiments, the fibroblasts are CD14, CD34, CD45, and/or HLA-DR negative. In some embodiments, the fibroblasts possess the ability to differentiate to osteogenic, chondrogenic, and adipogenic lineage cells.

Compositions of the present disclosure may be obtained from isolated cells or a population thereof capable of proliferating and differentiating into ectoderm, mesoderm, or endoderm. In some embodiments, an isolated fibroblast cell expresses at least one of Oct-4, Nanog, Sox-2, KLF4, c-Myc, Rex-1, GDF-3, LIF receptor, CD105, CD117, CD344 or Stella markers. In some embodiments, an isolated fibroblast cell does not express at least one of MHC class I, MHC class II, CD45, CD13, CD49c, CD66b, CD73, CD105, or CD90 cell surface proteins. Such isolated fibroblast cells may be used as a source of conditioned media. The cells may be cultured alone, or may by cultured in the presence of other cells in order to further upregulate production of growth factors in the conditioned media.

In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5, TrkA, BMP2, BMP4, FGF2, FGF4, PDGF, PGF, TGFα, TGFβ, and/or VEGF.

Cell populations may be expanded and utilized by administration themselves, or may be cultured in a growth media in order to obtain conditioned media. The term Growth Medium generally refers to a medium sufficient for the culturing of fibroblasts. In particular embodiments, medium for the culturing of the cells of the disclosure herein comprises Dulbecco's Modified Essential Media (DMEM). The media may comprise DMEM-low glucose (also DMEM-LG herein) (Invitrogen®, Carlsbad, Calif.). The DMEM-low glucose may be supplemented with 15% (v/v) fetal bovine serum (e.g. defined fetal bovine serum, Hyclone™, Logan Utah), antibiotics/antimycotics (such as penicillin (100 Units/milliliter), streptomycin (100 milligrams/milliliter), and amphotericin B (0.25 micrograms/milliliter), (Invitrogen®, Carlsbad, Calif.)), and 0.001% (v/v) 2-mercaptoethanol (Sigma®, St. Louis Mo.). In some cases different growth media are used, or different supplementations are provided, and these are normally indicated as supplementations to Growth Medium. Also relating to the present disclosure, the term standard growth conditions, as used herein refers to culturing of cells at 37° C., in a standard atmosphere comprising 5% CO₂, where relative humidity is maintained at about 100%. While the foregoing conditions are useful for culturing, it is to be understood that such conditions are capable of being varied by the skilled artisan who will appreciate the options available in the art for culturing cells, for example, varying the temperature, CO₂, relative humidity, oxygen, growth medium, and the like.

Also disclosed herein are cultured cells. Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time”.

Fibroblast cells used in the disclosed methods can undergo at least 25, 30, 35, or 40 doublings prior to reaching a senescent state. Methods for deriving cells capable of doubling to reach 10¹⁴ cells or more are provided. Examples are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less. In one embodiment, fibroblast cells used are isolated and expanded, and possess one or more markers selected from a group consisting of CD10, CD13, CD44, CD73, CD90, CD141, PDGFr-alpha, HLA-A, HLA-B, and HLA-C. In some embodiments, the fibroblast cells do not produce one or more of CD31, CD34, CD45, CD117, CD141, HLA-DR, HLA-DP, or HLA-DQ.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

In some cases, cell populations are obtained from a biopsy, and the donor providing the biopsy may be either the individual to be treated (autologous), or the donor may be different from the individual to be treated (allogeneic). In cases wherein allogeneic cells are utilized for an individual, the cells may come from one or a plurality of donors.

The fibroblasts may be fibroblasts obtained from various sources including, for example, dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and fibroblasts obtained from a plastic surgery-related by-product. In some embodiments, fibroblasts are dermal fibroblasts.

The fibroblasts utilized in certain embodiments of the disclosure may be generated by outgrowth from a biopsy of a subject's own skin (in the case of autologous preparations), or skin of healthy donors (for allogeneic preparations). In some embodiments, fibroblasts are used from young donors. In another embodiment, fibroblasts are transfected with genes to allow for enhanced growth and overcoming of the Hayflick limit. Subsequent to derivation of cells expansion in culture using standard cell culture techniques.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

In some embodiments, fibroblasts are transfected with one or more angiogenic genes to enhance ability to promote neural repair. An “angiogenic gene” describes a gene encoding for a protein or polypeptide capable of stimulating or enhancing angiogenesis in a culture system, tissue, or organism. Examples of angiogenic genes which may be useful in transfection of fibroblasts include activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin, fibronectin receptor, Factor X, HB-EGF, HBNF, HGF, HUAF, heart derived inhibitor of vascular cell proliferation, IL1, IGF-2 IFN-gamma, α1β1 integrin, α2β1 integrin, K-FGF, LIF, leiomyoma-derived growth factor, MCP-1, macrophage-derived growth factor, monocyte-derived growth factor, MD-ECI, MECIF, MMP2, MMP3, MMP9, urokiase plasminogen activator, neuropilin, neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-β, TGF-β receptors, TIMPs, TNF-α, transferrin, thrombospondin, urokinase, VEGF-A, VEGF-B, VEGF-C, VEGF-D, VEGF-E, VEGF, VEGF(164), VEGI, and EG-VEGF. Fibroblasts transfected with one or more angiogenic factors may be used in the disclosed methods of disease treatment or prevention.

Under appropriate conditions, fibroblasts may be capable of producing interleukin-1 (IL-1) and/or other inflammatory cytokines. In some embodiments, fibroblasts of the present disclosure are modified (e.g., by gene editing) to prevent or reduce expression of IL-1 or other inflammatory cytokines. For example, in some embodiments, fibroblasts are fibroblasts having a deleted or non-functional IL-1 gene, such that the fibroblasts are unable to express IL-1. Such modified fibroblasts may be useful in the therapeutic methods of the present disclosure by having limited pro-inflammatory capabilities when provided to a subject. In some embodiments, fibroblasts are treated with (e.g., cultured with) TNF-α, thereby inducing expression of growth factors and/or fibroblast proliferation.

In some embodiments, fibroblasts of the present disclosure are used as precursor cells that differentiate following introduction into an individual. In some embodiments, fibroblasts are subjected to differentiation into a different cell type (e.g., a hematopoietic cell) prior to introduction into the individual.

As disclosed herein, fibroblasts may secrete one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts of the present disclosure are cultured with an inhibitor of mRNA degradation. In some embodiments, fibroblasts are cultured under conditions suitable to support reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/L, 4.5 g/L, 4 g/L, 3 g/L, 2 g/L, or 1 g/L. In some embodiments, such conditions comprise glucose of about 1 g/L.

Aspects of the present disclosure comprise generating conditioned media from fibroblasts and utilization thereof. Conditioned medium may be obtained from culture with fibroblasts. The cells may be cultured for about 1, 2, 3, 4, 5, 6, 7, 8, 9, 10 days or more. In some embodiments, the fibroblasts are cultured for about 3 days prior to collecting conditioned media. Conditioned media may be obtained by separating the cells from the media. Conditioned media may be centrifuged (e.g., at 500× g). Conditioned media may be filtered through a membrane. The membrane may be a >1000 kDa membrane. Conditioned media may be subject to liquid chromatography such as HPLC. Conditioned media may be separated by size exclusion.

In some embodiments, the present disclosure utilizes exosomes derived from fibroblasts as a therapeutic modality. Exosomes derived from fibroblasts may be used in addition to, or in place of, fibroblasts in the various methods and compositions disclosed herein. Exosomes, also referred to as “microparticles” or “particles,” may comprise vesicles or a flattened sphere limited by a lipid bilayer. The microparticles may comprise diameters of 40-100 nm. The microparticles may be formed by inward budding of the endosomal membrane. The microparticles may have a density of about 1.13-1.19 g/mL and may float on sucrose gradients. The microparticles may be enriched in cholesterol and sphingomyelin, and lipid raft markers such as GM1, GM3, flotillin and the src protein kinase Lyn. The microparticles may comprise one or more proteins present in fibroblast, such as a protein characteristic or specific to the fibroblasts or fibroblast conditioned media. They may comprise RNA, for example miRNA. The microparticles may possess one or more genes or gene products found in fibroblasts or medium which is conditioned by culture of fibroblasts. The microparticles may comprise molecules secreted by the fibroblasts. Such a microparticle, and combinations of any of the molecules comprised therein, including in particular proteins or polypeptides, may be used to supplement the activity of, or in place of, the cell populations herein for the purpose of, for example, treating or preventing an autoimmune disease. The microparticle may comprise a cytosolic protein found in cytoskeleton e.g., tubulin, actin and actin-binding proteins, intracellular membrane fusions and transport, e.g., annexins and rab proteins, signal transduction proteins, e.g., protein kinases, 14-3-3 and heterotrimeric G proteins, metabolic enzymes, e.g., peroxidases, pyruvate and lipid kinases, and enolase-1 and the family of tetraspanins, e.g., CD9, CD63, CD81 and CD82. In particular, the microparticle may comprise one or more tetraspanins.

### IV. Fibroblast Isolation, Preparation, and Culturing

The following procedures demonstrate the combination of certain embodiments for obtaining and culturing fibroblasts but are not intended to be limiting as to the only procedures for obtaining fibroblasts for use in the embodiments disclosed herein. Skin tissue (dermis and epidermis layers) may be biopsied from a subject's post-auricular area. In one embodiment, the starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0+/−2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37+/−2.0° C. with 5.0+/−1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37+/−2.0° C. with 5.0+/−1.0% CO₂ and passaged every three to five days in the T-500 flask and every five to seven days in the ten layer cell stack (10CS). Cells should not remain in the T-500 flask for more than 10 days prior to passaging. Quality Control (QC) release testing for safety of the Bulk Drug Substance includes sterility and endotoxin testing. When cell confluence in the T-500 flask is approximately 95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37+/−2.0° C. with 5.0+/−1.0% CO₂ and fed with fresh Complete Growth Media every five to seven days. Cells should not remain in the 10CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

In some embodiments, when large numbers of cells are required after receiving cell count results from the primary 10 CS harvest, an additional passage into multiple cell stacks (up to four 10 CS) is performed. For additional passaging, cells from the primary harvest are added to a 2 L media bottle containing fresh Complete Growth Media. Resuspended cells are added to multiple cell stacks and incubated at 37+/−2.0° C. with 5.0+/−1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above. The method decreases or eliminates immunogenic proteins be avoiding their introduction from animal-sourced reagents. To reduce process residuals, cells are cryopreserved in protein-free freeze media, then thawed and washed prior to prepping the final injection to further reduce remaining residuals. If additional Drug Substance is needed after the harvest and cryopreservation of cells from additional passaging is complete, aliquots of frozen Drug Substance—Cryovial are thawed and used to seed 5 CS or 10 CS culture vessels. Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest.

At the completion of culture expansion, the cells are harvested and washed, then formulated to contain 1.0-2.7×10⁷ cells/mL, with a target of 2.2×10⁷ cells/mL. Alternatively, the target can be adjusted within the formulation range to accommodate different indication doses. The drug substance consists of a population of viable, autologous human fibroblast cells suspended in a cryopreservation medium consisting of Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. In addition to cell count and viability, purity/identity of the Drug Substance is performed and must confirm the suspension contains 98% or more fibroblasts. The usual cell contaminants include keratinocytes. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin β4 chain, is a 205 kDa transmembrane glycoprotein which associates with integrin α6 chain (CD49f) to form the α6/β4 complex. This complex has been shown to act as a molecular marker for keratinocyte cells (Adams and Watt 1991).

Antibodies to CD 104 protein bind to 100% of human keratinocyte cells. Cell count and viability is determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead. The method was custom developed specifically for use in determining purity/identity of autologous cultured fibroblasts. Alternatively, cells can be passaged from either the T-175 flask (or alternatives) or the T-500 flask (or alternatives) into a spinner flask containing microcarriers as the cell growth surface. Microcarriers are small bead-like structures that are used as a growth surface for anchorage dependent cells in suspension culture. They are designed to produce large cell yields in small volumes. In this apparatus, a volume of Complete Growth Media ranging from 50 mL-300 mL is added to a 500 mL, IL or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37+/−2.0° C. with 5.0+/−1.0% CO₂ incubator to allow for adherence of cells to the carriers. After the attachment period, the speed of the spin plate is increased (30-120 RPM). Cells are fed with fresh Complete Growth Media every one to five days, or when media appears spent by color change. Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsiziation and reseeding. Alternatively, if enough cells are produced from the initial T-175 or T-500 flask, the cells can be directly seeded into the scale-up amount of microcarriers. After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA. Microcarriers used within the disposable spinner flask may be made from poly blend such as BioNOC II® (Cesco Bioengineering, distributed by Bellco Biotechnology, Vineland, N.J.) and FibraCel® (New Brunswick Scientific, Edison, N.J.), gelatin, such as Cultispher-G (Percell Biolytica, Astrop, Sweden), cellulose, such as Cytopore™ (GE Healthcare, Piscataway, N.J.) or coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah).

In an embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Certain embodiments concern enzyme activites selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease may be used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion may be serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain embodiments, a mixture of collagenase and the neutral protease dispase are used in the dissociating step. Certain embodiments concern methods which employ digestion in the presence of at least one collagenase from Clostridium histolyticum, and either of the protease activities, dispase and thermolysin. Certain embodiments concern methods employing digestion with both collagenase and dispase enzyme activities. Certain embodiments concern methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the disclosure. Preferred enzyme treatments are 0.5, 1, 1.5, or 2 hours long or longer. In certain embodiments, the tissue is incubated at 37° C. during the enzyme treatment of the dissociation step. Diluting the digest may also improve yields of cells as cells may be trapped within a viscous digest.

While the use of enzyme activities may be used, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. The culture temperature may be 37° C., however the temperature may range from about 35° C. to 39° C. depending on the other culture conditions and desired use of the cells or culture.

Certain embodiments concern methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Certain embodiments concern factors to be added for growth on serum-free media include one or more of FGF, EGF, IGF, and PDGF. In some embodiments, two, three or all four of the factors are add to serum free or chemically defined media. In other embodiments, LIF is added to serum-free medium to support or improve growth of the cells.

### V. Administration of Therapeutic Compositions

The therapy provided herein may comprise administration of a therapeutic agents (e.g., cell populations encompassed herein) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., a cell population) of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 mg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

### VI. Kits of the Disclosure

Any of the cellular and/or non-cellular compositions described herein or similar thereto may be comprised in a kit. In a non-limiting example, one or more reagents for use in methods for preparing fibroblasts, fibroblast-derived products, or derivatives thereof (e.g., exosomes derived from fibroblasts), immune cells, generation thereof, or a combination thereof may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and may be suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### Examples

The following examples are included to demonstrate particular embodiments of the disclosure. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Fibroblasts Generate CD25+ Immune Modulatory Cells

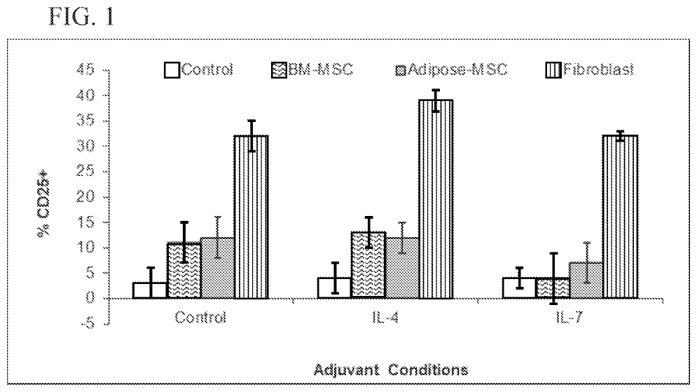

Fibroblasts, bone marrow mesenchymal stem cells (BM-MSC), and adipose mesenchymal stem cells (Adipose-MSC) were co-cultured with immune cells alone or in the presence of IL-4 or IL-7. The percentage of cells expressing CD25 were measured as shown in FIG. 1.

### Example 2: IL-2 Enhances CD25+ Cell Generation by Fibroblasts

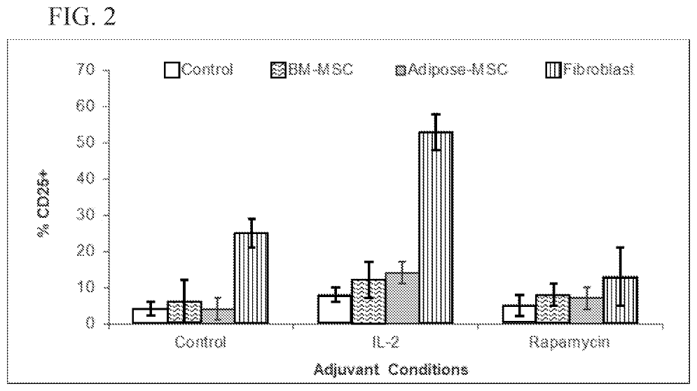

Fibroblasts, bone marrow mesenchymal stem cells (BM-MSC), and adipose mesenchymal stem cells (Adipose-MSC) were co-cultured with immune cells alone or in the presence of IL-2 or rapamycin. The percentage of cells expressing CD25 were measured as shown in FIG. 2.

### Example 3: IL-2 Enhances CD25+ Cell Generation by Fibroblasts

Fibroblasts, bone marrow mesenchymal stem cells (BM-MSC), and adipose mesenchymal stem cells (Adipose-MSC) were co-cultured with immune cells alone or in the presence of IL-2 or rapamycin. The percentage of generated Treg cells expressing FoxP3 were measured as shown in FIG. 2.

### Example 4: IL-2 and Fibroblast-Generated Treg Suppress T Cell Proliferation

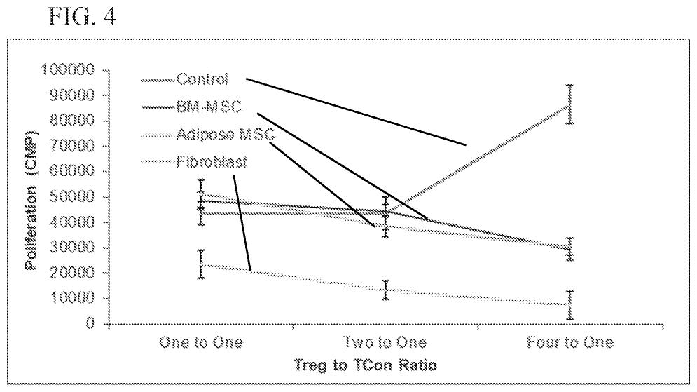

Fibroblasts, bone marrow mesenchymal stem cells (BM-MSC), and adipose mesenchymal stem cells (Adipose-MSC) were co-cultured with Treg cells at the provided ratios shown in FIG. 4. Proliferation for each condition was measured as shown in FIG. 4.

### Example 5: IL-2 and Fibroblast-Generated Treg Suppress T Cell Proliferation

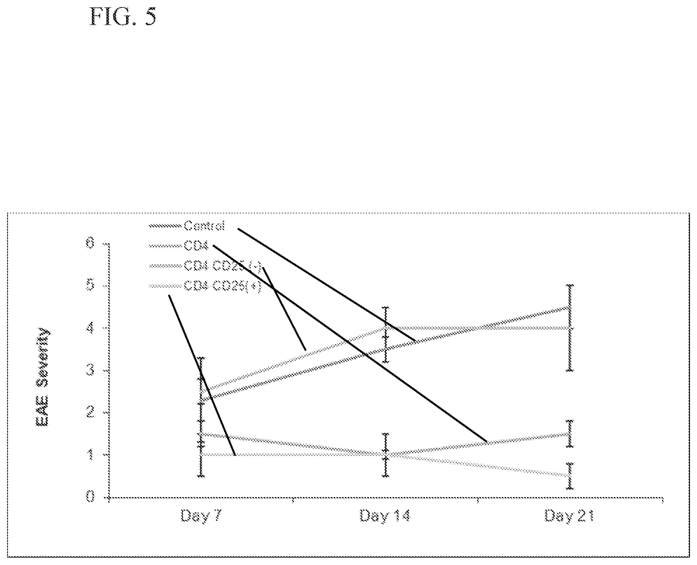

Tregs generated from the co-culture with fibroblasts and IL-2 were assessed for the EAE severity after 7, 14, and 21 days as shown in FIG. 5.

### REFERENCES

All patents and publications mentioned in the specification are indicative of the level of those skilled in the art to which the disclosure pertains. All patents and publications are herein incorporated by reference to the same extent as if each individual publication was specifically and individually indicated to be incorporated by reference.
1. Adams, N. M., et al., NK Cell Responses Redefine Immunological Memory. J Immunol, 2016. 197(8): p. 2963-2970.
2. Wang, X. Y., et al., LncRNA ROR1-AS1 promotes colon cancer cell proliferation by suppressing the expression of DUSP5/CDKN1A. Eur Rev Med Pharmacol Sci, 2020. 24(3): p. 1116-1125.
3. Serre, L., N. Fazilleau, and S. Guerder, Central tolerance spares the private high-avidity CD4(+) T-cell repertoire specific for an islet antigen in NOD mice. Eur J Immunol, 2015. 45(7): p. 1946-56.
4. Enouz, S., et al., Autoreactive T cells bypass negative selection and respond to self-antigen stimulation during infection. J Exp Med, 2012. 209(10): p. 1769-79.
5. Aluvihare, V. R., M. Kallikourdis, and A. G. Betz, Regulatory T cells mediate maternal tolerance to the fetus. Nat Immunol, 2004. 5(3): p. 266-71.
6. Somerset, D. A., et al., Normal human pregnancy is associated with an elevation in the immune suppressive CD25+CD4+ regulatory T-cell subset. Immunology, 2004. 112(1): p. 38-43.
7. Campos-Mora, M., et al., CD4+Foxp3+T Regulatory Cells Promote Transplantation Tolerance by Modulating Effector CD4+ T Cells in a Neuropilin-1-Dependent Manner. Front Immunol, 2019. 10: p. 882.
8. Lalfer, M., et al., Foxp3(+) Regulatory and Conventional CD4(+) T Cells Display Similarly High Frequencies of Alloantigen-Reactive Cells. Front Immunol, 2019. 10: p. 521.
9. Wang, K., et al., Different phenotypes of CD4(+)CD25(+)Foxp3(+) regulatory T cells in recipients post liver transplantation. Int Immunopharmacol, 2019. 69: p. 194-201.
10. Herrera-Gomez, F., et al., The number of FoxP3 regulatory T cells in the circulation may be a predictive biomarker for kidney transplant recipients: A multistage systematic review. Int Immunopharmacol, 2018. 65: p. 483-492.
11. Leung, C. S., et al., Single-cell transcriptomics reveal that PD-1 mediates immune tolerance by regulating proliferation of regulatory T cells. Genome Med, 2018. 10(1): p. 71.
12. Zhu, X., et al., CD4+CD25+Tregs express an increased LAG-3 and CTLA-4 in anterior chamber-associated immune deviation. Graefes Arch Clin Exp Ophthalmol, 2007. 245(10): p. 1549-57.
13. Zhang, H., et al., Involvement of Foxp3-expressing CD4+CD25+ regulatory T cells in the development of tolerance induced by transforming growth factor-beta2-treated antigen presenting cells. Immunology, 2008. 124(3): p. 304-14.
14. He, H., et al., Upregulation of CD94 on CD8+ T cells in anterior chamber-associated immune deviation. BMC Immunol, 2008. 9: p. 53.
15. Saban, D. R., et al., The role of ACAID and CD4+CD25+FOXP3+ regulatory T cells on CTL function against MHC alloantigens. Mol Vis, 2008. 14: p. 2435-42.
16. Hori, J., et al., GITR ligand-mediated local expansion of regulatory T cells and immune privilege of corneal allografts. Invest Ophthalmol Vis Sci, 2010. 51(12): p. 6556-65.
17. Ji, S. X., X. L. Yin, and P. Z. Yang, Effect of CD4(+)CD25(+) regulatory T cells in the development of anterior chamber-associated immune deviation. Int J Ophthalmol, 2011. 4(1): p. 19-25.
18. Zhang, Y., et al., Effects of human umbilical cord-derived mesenchymal stem cells on anterior chamber-associated immune deviation. Int Immunopharmacol, 2013. 15(1): p. 114-20.
19. Yan, F., et al., Tolerogenic dendritic cells suppress murine corneal allograft rejection by modulating CD28/CTLA-4 expression on regulatory T cells. Cell Biol Int, 2014. 38(7): p. 835-48.
20. Wang, T., et al., c-Rel is Required for the Induction of pTregs in the Eye but Not in the Gut Mucosa. Immunol Invest, 2016. 45(8): p. 776-786.
21. Kunishige, T., et al., Protective Role of ICOS and ICOS Ligand in Corneal Transplantation and in Maintenance of Immune Privilege. Invest Ophthalmol Vis Sci, 2016. 57(15): p. 6815-6823.
22. Zenclussen, A. C., et al., Abnormal T-cell reactivity against paternal antigens in spontaneous abortion: adoptive transfer of pregnancy-induced CD4+CD25+T regulatory cells prevents fetal rejection in a murine abortion model. Am J Pathol, 2005. 166(3): p. 811-22.
23. Zhao, T. X., et al., Low-dose interleukin-2 in patients with stable ischaemic heart disease and acute coronary syndromes (LILACS): protocol and study rationale for a randomised, double-blind, placebo-controlled, phase I/II clinical trial. BMJ Open, 2018. 8(9): p. e022452.
24. Jyonouchi, S., et al., Phase I trial of low-dose interleukin 2 therapy in patients with Wiskott-Aldrich syndrome. Clin Immunol, 2017. 179: p. 47-53.
25. Asano, T., et al., Phase I/Ha Study of Low Dose Subcutaneous Interleukin-2 (IL-2) for Treatment of Refractory Chronic Graft Versus Host Disease. Acta Med Okayama, 2016. 70(5): p. 429-433.
26. Kennedy-Nasser, A. A., et al., Ultra low-dose IL-2 for GVHD prophylaxis after allogeneic hematopoietic stem cell transplantation mediates expansion of regulatory T cells without diminishing antiviral and antileukemic activity. Clin Cancer Res, 2014. 20(8): p. 2215-25.
27. Mizui, M. and G. C. Tsokos, Low-Dose IL-2 in the Treatment of Lupus. Curr Rheumatol Rep, 2016. 18(11): p. 68.
28. Todd, J. A., et al., Regulatory T Cell Responses in Participants with Type 1 Diabetes after a Single Dose of Interleukin-2: A Non-Randomised, Open Label, Adaptive Dose-Finding Trial. PLoS Med, 2016. 13(10): p. e1002139.
29. Pham, M. N., M. G. von Herrath, and J. L. Vela, Antigen-Specific Regulatory T Cells and Low Dose of IL-2 in Treatment of Type 1 Diabetes. Front Immunol, 2015. 6: p. 651.
30. Waldron-Lynch, F., et al., Rationale and study design of the Adaptive study of IL-2 dose on regulatory T cells in type 1 diabetes (DILT1D): a non-randomised, open label, adaptive dose finding trial. BMJ Open, 2014. 4(6): p. e005559.
31. Wei, C., et al., 1-Methyl-tryptophan attenuates regulatory T cells differentiation due to the inhibition of estrogen-IDO1-MRC2 axis in endometriosis. Cell Death Dis, 2016. 7(12): p. e2489.

What is claimed is:

1. A method of generating an immune modulatory cell population comprising the step of:
contacting a first cell population with a second cell population, wherein the first cell population comprises a fibroblast population, wherein the second cell population comprises an immune cell population obtained from a subject in need of therapy,
wherein the contacting generates a third cell population, wherein the cell population has immune modulatory properties.

2. The method of claim 1, wherein the fibroblast population comprises a fibroblast population isolated from tissue selected from the group consisting of bone marrow, adipose, placenta, tonsil, skin, hair follicle, omentum, peripheral blood, wharton's jelly, and a combination thereof.

3. The method of claim 1 or 2, wherein the fibroblast population is allogeneic to the subject.

4. The method of any one of claims 1-3, wherein the fibroblast population expresses at least one marker selected from the group consisting of CD13, CD44, CD49b, CD73, CD90, CD105, and a combination thereof.

5. The method of any one of claims 1-4, wherein the fibroblast population lacks expression of at least one marker selected from the group consisting of: CD14, CD34, CD45, HLA class II, SSEA-1, and a combination thereof.

6. The method of any one of claims 1-5, wherein the fibroblast population comprises cells capable of differentiating into bone, cartilage, and/or adipose tissue.

7. The method of any one of claims 1-6, wherein the fibroblast population is contacted with hCG prior to contacting the fibroblast population with the second cell population.

8. The method of claim 7, wherein the fibroblast population is contacted with hCG at a concentration of 5 IU/mL.

9. The method of any one of claims 1-8, wherein the fibroblast population produces FGF-1 at a concentration of at least 2 ng/mL per million cells.

10. The method of any one of claims 1-9, wherein the fibroblast population produces FGF-2 at a concentration of at least 1 ng/mL per million cells.

11. The method of claim 1, wherein the fibroblast population produces TGF-beta at a concentration of at least 10 ng/mL per million cells

12. The method of any one of claims 1-11, wherein the fibroblast population produces exosomes at a concentration of at least 10 ng/mL per million cells

13. The method of claim 12, wherein said exosomes comprise the marker CD81.

14. The method of claim 12, wherein said exosomes comprise membrane bound TGF-beta.

15. The method of any one of claims 1-14, wherein the fibroblast population comprises a cell population derived from Wharton's Jelly that has been enzymatically digested and the resulting cells have been centrifuged, plated onto fibronectin-coated plates in medium with 2% serum, wherein the fibroblast population was selected from cell colonies that adhere to the plates, and optionally wherein the fibroblast population has mortal, epithelioid morphology.

16. The method of any one of claims 1-15, wherein the fibroblast population possesses an ability to inhibit secretion of at least one inflammatory cytokine.

17. The method of claim 16, wherein the inflammatory cytokine is selected from the group consisting of IFN-gamma, TNF-alpha, IL-2, IL-7, IL-12, IL-15, IL-17, IL-18, IL-21, IL-23, IL-27, IL-33, HMGB-1, TRAIL, and a combination thereof.

18. The method of any one of claims 1-18, wherein the fibroblast population is cultured for at least 14 days.

19. The method of any one of claims 1-19, wherein the fibroblast population is maintained in an undifferentiated state when contacted with the second population.

20. The method of any one of claims 1-20, wherein the fibroblast population is cultured in the presence of nerve growth factor, bFGF, dibutryl cAMP, IBMX, and/or retinoic acid for approximately, at least, or at most four weeks.

21. The method of any one of claims 1-21, wherein said immune cell population comprises immune cells selected from the group consisting of B cells, T cells, innate lymphoid cells, natural killer cells, natural killer T cells, gamma delta T cells, macrophages, monocytes, dendritic cells, neutrophils, myeloid derived suppressor cells, and a combination thereof.

22. The method of claim 21, wherein said B cells comprise CD5+ B cells and/or CD5− B cells.

23. The method of claim 21, wherein said B cells comprise naïve B cells and/or memory B cells.

24. The method of claim 21, wherein said T cells comprise CD4+ and/or CD8+ T cells.

25. The method of claim 21, wherein said T cells comprise CD28+ and/or CD28− T cells.

26. The method of claim 21, wherein said T cells comprise memory T cells and/or naïve T cells.

27. The method of claim 21, wherein said T cells express CD3.

28. The method of claim 21, wherein said T cells comprise T regulatory cells.

29. The method of claim 28, wherein said T regulatory cells express a protein selected from the group consisting of CD25, CTLA-4, FoxP3, and a combination thereof.

30. The method of claim 21, said innate lymphoid cells are selected from the group consisting of innate lymphoid cells 1, innate lymphoid cells 2, innate lymphoid cells 3, lymphoid tissue inducer cells, and a combination thereof.

31. The method of claim 30, wherein said innate lymphoid cell 1 expresses T bet, responds to IL-12 by secretion of interferon gamma, and/or lacks expression of perforin and CD56.

32. The method of claim 30, wherein said innate lymphoid cell 2 produces IL-4 and/or IL-13.

33. The method of claim 30, wherein said innate lymphoid cell 3 produces IL-17a and/or IL-22.

34. The method of claim 30, wherein said lymphoid tissue inducer cells comprise cells involved in the induction of memory T cells.

35. The method of claim 21, wherein said T cells comprise Th1 cells.

36. The method of claim 35, wherein said Th1 cells are capable of secreting cytokines selected from the group consisting of interferon gamma, interleukin 2, TNF-beta.

37. The method of claim 36, wherein said Th1 cells express markers selected from the group consisting of CD4, CD94, CD119 (IFNγ R1), CD183 (CXCR3), CD186 (CXCR6), CD191 (CCR1), CD195 (CCR5), CD212 (IL-12Rβ1&2), CD254 (RANKL), CD278 (ICOS), IL-18R,MRP1, NOTCH3, TIM3, and a combination thereof.

38. The method of claim 21, wherein said T cells comprise Th2 cells.

39. The method of claim 38, wherein said Th2 cells are capable of secreting cytokines selected from the group consisting of IL-4, IL-5, IL-6, IL-9, IL-10, IL-13, and a combination thereof.

40. The method of claim 38, wherein said Th2 cells express markers selected from the group consisting of CRTH2, CCR4, CCR3, and a combination thereof.

41. The method of claim 21, wherein said macrophages comprise M1 macrophages and/or M2 macrophages.

42. The method of claim 41, wherein said M1 macrophages are capable of producing nitric oxide.

43. The method of claim 41, wherein said M2 macrophages are capable of producing arginase.

44. The method of claim 21, wherein said dendritic cells comprise myeloid dendritic cells or lymphoid dendritic cells.

45. The method of claim 21, wherein said myeloid dendritic cells are capable of stimulating Th1 immune responses in a mature state.

46. The method of claim 45, wherein said myeloid dendritic cells in said mature state express substantially higher levels of CD80 as compared to myeloid dendritic cells in an immature state.

47. The method of claim 45, wherein said myeloid dendritic cells in said mature state express substantially higher levels of CD86 as compared to myeloid dendritic cells in an immature state.

48. The method of claim 45, wherein said myeloid dendritic cells in said mature state express substantially higher levels of CD40 as compared to myeloid dendritic cells in an immature state.

49. The method of claim 44, wherein said lymphoid dendritic cells are capable of producing interferon alpha.

50. The method of any one of claims 1-49, wherein the second cell population comprises peripheral blood mononuclear cells.

51. The method of any one of claims 1-50, wherein the second cell population comprises buffy coat cells.

52. The method of any one of claims 1-52, wherein the second cell population is cultured with amniotic fluid stem cells in a manner permitting cell to cell contact.

53. The method of claim 52, wherein said cell to cell contact is provided by culturing on tissue culture plates at a concentration sufficient to cover a surface of said tissue culture plate.

54. The method of claim 52, wherein the ratio of cells of the second cell population to amniotic fluid stem cells is 1 to 1 when the second cell population comprises peripheral blood mononuclear cells.

55. The method of claim 53, wherein the ratio of cells of the second cell population to amniotic fluid stem cells is 1 to 5 when the second cell population comprises T regulatory cell.

56. The method of any one of claims 1-55, wherein the second cell population comprises CD3 isolated T cells which have been in a tissue culture at a 1:2 ratio with amniotic fluid stem cells in the presence of interleukin-2 for approximately 7 days.

57. The method of claim 56, wherein said interleukin-2 is added to the tissue culture at a concentration sufficient to induce expansion of T cells with immune modulatory activity.

58. The method of claim 57, wherein said concentration of interleukin-2 is 2 pg/mL to 100 ng/mL of tissue culture fluid.

59. The method of any one of claims 1-58, wherein the second cell population comprises CD3 isolated T cells which have been cultured at a 1:2 ratio with amniotic fluid stem cells in the presence of VEGF for approximately 7 days.

60. The method of any one of claims 1-59, wherein the second cell population comprises CD3 isolated T cells which have been cultured at a 1:2 ratio with amniotic fluid stem cells in the presence of PGE-2 for approximately 7 days.

61. The method of any one of claims 1-60, wherein the second cell population comprises CD3 isolated T cells which have been cultured at a 1:2 ratio with amniotic fluid stem cells in the presence of TGF-beta for approximately 7 days.

62. The method of any one of claims 1-61, wherein the second cell population comprises CD3 isolated T cells which have been cultured at a 1:2 ratio with amniotic fluid stem cells in the presence of an mTOR inhibitor for approximately 7 days.

63. The method of claim 62, wherein said mTOR inhibitor comprises rapamycin.

64. The method of claim 62, wherein said mTOR inhibitor comprises everolimus.

65. The method of any one of claims 1-64, wherein the second cell population comprises CD3 isolated T cells which have been cultured at a 1:2 ratio with amniotic fluid stem cells in the presence of anti-CD3 and anti-CD28 antibodies for approximately 7 days.

66. The method of claim 65, wherein said anti-CD3 and anti-CD28 antibodies have been immobilized to a solid surface during the tissue culture.

67. The method of claim 65, wherein said solid surface is a tissue culture plate.

68. The method of claim 65, wherein said solid surface is a biocompatible bead.

69. The method of any one of claims 1-68, further comprising adding interleukin-2 during the contacting step to allow for expansion of T cells with immune regulatory properties.

70. The method of any one of claims 1-69, wherein the subject in need of therapy has an autoimmune condition.

71. The method of claim 70, wherein the autoimmune condition is multiple sclerosis.
